(19) United States
(12) Patent Application Publication
(10) Pub. No.: US 2022/0025353 A1
(43) Pub. Date: Jan. 27, 2022

(54) NANOPORE MEMBRANE DEVICE AND METHODS OF USE THEREOF

(71) Applicant: The Regents of the University of California, Oakland, CA (US)

(72) Inventors: Jennifer A. Doudna, Berkeley, CA (US); Yuhong Cao, Berkeley, CA (US); Enbo Ma, Moraga, CA (US); Peidong Yang, Berkeley, CA (US)

(21) Appl. No.: 17/277,652

(22) PCT Filed: Sep. 26, 2019
(86) PCT No.: PCT/US19/53154

§ 371 Date: Mar. 18, 2021

Related U.S. Application Data
(60) Provisional application No. 62/738,920, filed on Sep. 28, 2018.

(51) Int. Cl. C12N 13/00; C12N 15/87; C12N 15/11; C12N 9/22

(52) U.S. Cl. CPC C12N 13/00; C12N 15/87; B82Y 5/00; C12N 9/22; C12N 2310/20; C12N 15/111

(57) ABSTRACT

The present disclosure provides devices and methods for delivering a biomolecule into a cell. A delivery device of the present disclosure includes a first reservoir, a second reservoir, a porous membrane comprising a nanopore, and two or more electrodes configured to generate an electric field across the porous membrane for delivery of a biomolecule present in the second reservoir through the nanopore of the porous membrane and into a cell present in the first reservoir.

### CROSS-REFERENCE

This application claims the benefit of U.S. Provisional Patent Application No. 62/738,920, filed Sep. 28, 2018, which application is incorporated herein by reference in its entirety.

### INTRODUCTION

Several chemical, physical, and biological techniques have been used for delivering macromolecules into living cells. Delivery of biomolecules into living cells is essential for biomedical research and drug development as well as genome editing. However, conventional methods of delivery of biomolecules such as viral vectors, cell penetrating peptides, cationic lipids, positive charged polymers, bulk electroporation, and microinjection pose several challenges. Such challenges include safety concerns, toxicity, damage to the cells, limited loading capacity, low delivery efficiencies, low cell viabilities, low cell throughput, high cellular perturbation, and high costs.

There is a need in the art for delivery devices and methods that allow for permeabilization of the cell membrane to facilitate delivery of biomolecules into cells.

### SUMMARY

The present disclosure provides devices and methods for delivering a biomolecule into a cell. A delivery device of the present disclosure includes a first reservoir, a second reservoir, a porous membrane comprising a nanopore, and two or more electrodes configured to generate an electric field across the porous membrane for delivery of a biomolecule present in the second reservoir through the nanopore of the porous membrane and into a cell present in the first reservoir.

In one aspect, provided herein is a device for delivering a biomolecule into a eukaryotic cell, the device comprising: a first reservoir comprising a proximal end and a distal end: a second reservoir comprising a proximal end and a distal end; a porous membrane comprising at least one nanopore with a pore size ranging from about 50 nm to about 150 nm, wherein the at least one nanopore fluidically connects the first reservoir and the second reservoir; and two or more electrodes configured to generate an electric field from the second reservoir to the first reservoir.

### BRIEF DESCRIPTION OF THE DRAWINGS

FIGS. 1A-1C show a schematic of a delivery device of the present disclosure.

FIGS. 2A-2C show mRNA transfection of HEK293 (FIG. 2A), HeLa (FIG. 2B), and 3T3 (FIG. 2C) cells with a delivery device of the present disclosure with different voltage intensities.

FIGS. 3A-3C show DNA plasmid transfection of HEK293 (FIG. 3A), HeLa (FIG. 3B), and 3T3 (FIG. 3C) cells with the delivery device of the present disclosure with different voltage intensities.

FIG. 4 shows DNA plasmid transfection efficiencies of a delivery device of the present disclosure with different voltage intensities compared to Lipofectamine (LFN)-mediated delivery.

FIGS. 5A-5B show mRNA (FIG. 5A) and DNA plasmid (FIG. 5B) transfection using a delivery device of the present disclosure with different voltage intensities.

FIG. 6 shows DNA plasmid transfection efficiencies of a delivery device of the present disclosure with different voltage intensities compared to Lipofectamine-mediated delivery.

FIG. 7 shows a delivery device of the present disclosure in delivering mCherry-tagged STIM1 protein into HEK293 cells.

FIG. 8 shows T7E1 assays of HEK293 cells from transfection of Cas9 RNP with a delivery device of the present disclosure.

FIGS. 9A-9B show a toxicity comparison between a delivery device of the present disclosure and Lipofectamine 2000.

FIG. 10 provides a schematic depiction of a delivery device of the present disclosure.

### DEFINITIONS

As used herein, the term “nanopore” a nanoscale passageway in a membrane through which liquid, air, ionic current, biomolecules, etc. can flow.

As used herein, the term “plurality” contains at least 2 members. In certain cases, a plurality may have at least 10, at least 100, at least 10³, at least 10⁴, at least 10⁵, at least 10⁶, at least 10⁷, at least 10⁸ or at least 10⁹ or more members.

The term “naturally-occurring” or “unmodified” as used herein as applied to a nucleic acid, a polypeptide, a cell, or an organism, refers to a nucleic acid, polypeptide, cell, or organism that is found in nature. For example, a polypeptide or polynucleotide sequence that is present in an organism (including viruses) that can be isolated from a source in nature and which has not been intentionally modified by a human in the laboratory is naturally occurring.

The terms “peptide,” “polypeptide,” and “protein” are used interchangeably herein, and refer to a polymeric form of amino acids of any length, which can include coded and non-coded amino acids, chemically or biochemically modified or derivatized amino acids, and polypeptides having modified peptide backbones.

The terms “polynucleotide” and “nucleic acid,” used interchangeably herein, refer to a polymeric form of nucleotides of any length, either ribonucleotides or deoxyribonucleotides. Thus, this term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases. “Oligonucleotide” generally refers to polynucleotides of between about 5 and about 100 nucleotides of single- or double-stranded DNA. However, for the purposes of this disclosure, there is no upper limit to the length of an oligonucleotide. Oligonucleotides are also known as “oligomers” or “oligos” and may be isolated from genes, or chemically synthesized by methods known in the art. The terms “polynucleotide” and “nucleic acid” should be understood to include, as applicable to the embodiments being described, single-stranded (such as sense or antisense) and double-stranded polynucleotides.

As used herein, the term “polymer” refers to any compound that is made up of two or more monomeric units covalently bonded to each other, where the monomeric units may be the same or different, such that the polymer may be a homopolymer or a heteropolymer. Representative polymers include peptides, polysaccharides, nucleic acids and the like, where the polymers may be naturally occurring or synthetic.

As used herein, the term “biopolymer” refers to a polymer of one or more types of repeating units. Biopolymers are typically found in biological systems and particularly include polysaccharides (such as carbohydrates), and peptides (which term is used to include polypeptides and proteins) and polynucleotides as well as their analogs such as those compounds composed of or containing amino acid analogs or non-amino acid groups, or nucleotide analogs or non-nucleotide groups. This includes polynucleotides in which the conventional backbone has been replaced with a non-naturally occurring or synthetic backbone, and nucleic acids (or synthetic or naturally occurring analogs) in which one or more of the conventional bases has been replaced with a group (natural or synthetic) capable of participating in Watson-Crick type hydrogen bonding interactions.

As used herein, the term “fluorophore” refers to a molecule exhibiting specific fluorescence emission when excited by energy from an external source”. The terms “fluorescent dye”, “fluorescence dye” and “fluorophore” may be used interchangeably.

As used herein, the term “dye” or “stain” refers to a molecule having large absorptivity or high fluorescence quantum yield, and which demonstrates affinity for certain materials or cellular structures.

As used herein, the term “labeled” refers to means that carry one or more moiety/moieties that enable(s) the detection thereof. As used herein, the terms “label”, “detectable moiety” and “marker” may be used interchangeably.

As used herein, the term “luminescent dye” refers to every molecule that emits light upon a chemical or a biochemical reaction.

Before the present invention is further described, it is to be understood that this invention is not limited to particular cases described, as such may, of course, vary. It is also to be understood that the terminology used herein is for the purpose of describing particular cases only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “an,” and “the” include plural referents unless the context clearly dictates otherwise. Thus, for example, reference to “a eukaryotic cell” includes a plurality of such eukaryotic cells and reference to “the biomolecule” includes reference to one or more biomolecules and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the invention, which are, for clarity, described in the context of separate cases, may also be provided in combination in a single embodiment. Conversely, various features of the invention, which are, for brevity, described in the context of a single embodiment, may also be provided separately or in any suitable sub-combination. All combinations of the cases pertaining to the invention are specifically embraced by the present invention and are disclosed herein just as if each and every combination was individually and explicitly disclosed. In addition, all sub-combinations of the various cases and elements thereof are also specifically embraced by the present invention and are disclosed herein just as if each and every such sub-combination was individually and explicitly disclosed herein.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DETAILED DESCRIPTION

The present disclosure provides devices and methods for delivering a biomolecule into a cell. A delivery device of the present disclosure includes a first reservoir, a second reservoir, a porous membrane comprising a nanopore, and two or more electrodes configured to generate an electric field across the porous membrane for delivery of a biomolecule present in the second reservoir through the nanopore of the porous membrane and into a cell present in the first reservoir.

### Delivery Devices

Aspects of the present disclosure include a delivery device for transporting a biomolecule across a plasma membrane and into a cell.

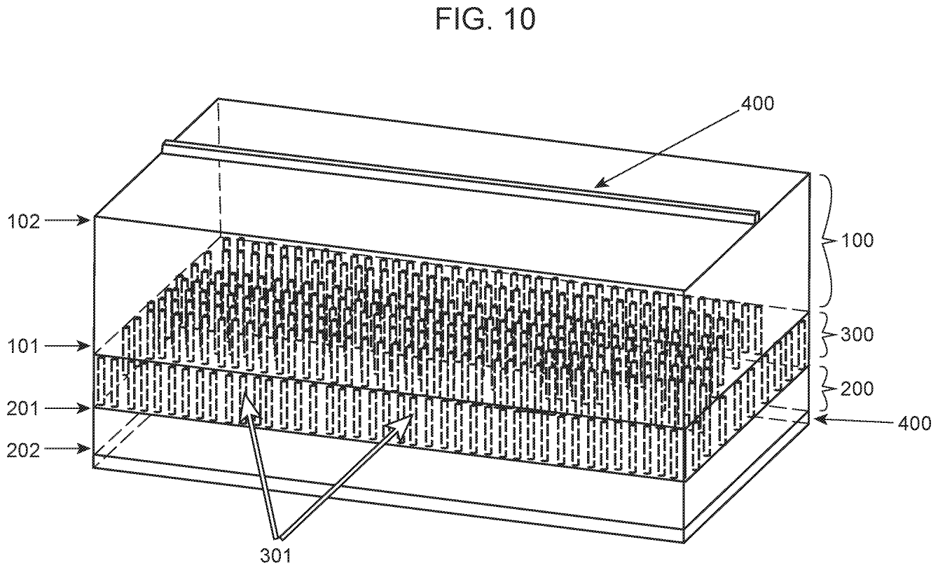

With reference to FIG. 10, the delivery device of the present disclosure includes a first reservoir 100 comprising a proximal end 101 and a distal end 102; a second reservoir 200 comprising a proximal end 201 and a distal end 202; a porous membrane 300 comprising at least one nanopore 301; and at least two electrodes 400.

In some cases, the first reservoir is formed from a cell culture dish, a cell culture plate, and/or a cell culture flask. In some cases, the first reservoir is formed from a material selected from a polystyrene (PS), polyethylene, polycarbonate, polyolefin, ethylene vinyl acetate, polypropylene, polysulfone, polytetrafluoroethylene (PTFE), a silicone rubber or copolymer, poly(styrene-butadiene-styrene), polydimethylsiloxane (PDMS)), polyimide, polyurethane, SU-8, polymethylmethacrylate (PMMA), polyethylene terephthalate (PET), polyvinylchloride (PVC)), polycaprolactone (PCL), or any combination thereof. SU-8 formulations comprise a monomer, containing epoxy moieties, a solvent, and a photoacid initiator. In some cases, the solvent in the SU-8 formulation is a cyclopentanone. In some cases, the photoacid initiator in the SU-8 formulation is a triarylsulfonium hexafluoroantimonate. On exposure to UV radiation, a photoacid is produced that protonates the epoxy moieties, which then react with neutral epoxy groups on heating, resulting in a cross-linked polymer network of high mechanical strength and thermal stability. See e.g. Nemani et al. 2013, Mater Sci Eng C Mater Biol Appl. 33(7): 10.1016, which is hereby incorporated by reference in its entirety.

In some cases, the first reservoir is formed from a material selected from a biocompatible polymer. Biocompatible polymers include natural or synthetic polymers. Non-limiting examples of biocompatible polymers include, but are not limited to poly(alpha esters) such as poly(lactate acid), poly(glycolic acid), polyorthoesters and polyanhydrides and their copolymers, polyglycolic acid and polyglactin, cellulose ether, cellulose, cellulosic ester, fluorinated polyethylene, phenolic, poly-4-methylpentene, polyacrylonitrile, polyamide, polyamideimide, polyacrylate, polybenzoxazole, polycarbonate, polycyanoarylether, polyester, polyestercarbonate, polyether, polyetheretherketone, polyetherimide, polyetherketone, polyethersulfone, polyethylene, polyfluoroolefin, polyimide, polyolefin, polyoxadiazole, polyphenylene oxide, polyphenylene sulfide, polypropylene, polystyrene, polysulfide, polysulfone, polytetrafluoroethylene, polythioether, polytriazole, polyurethane, polyvinyl, polyvinylidene fluoride, regenerated cellulose, silicone, urea-formaldehyde, polyglactin, or copolymers or a combination of these materials.

In some cases, the first reservoir is a cylindrical shape, a circular shape, a square shape, a spherical shape, cylindrical shape, or a rectangular shape. In some cases, the first reservoir includes walls that form the side boundary of the first reservoir. In some cases, the first reservoir is a first chamber. In some cases, the first reservoir is integral and/or included with a porous membrane. In some cases, the first reservoir is integral and/or included with a second reservoir. In some cases, the first reservoir is integral and/or included with a second reservoir and a porous membrane. In some cases, the first reservoir is separate e.g. reversibly disconnectable or detachable, from a second reservoir. In some cases, the second reservoir is reversibly disconnectable or detachable from the porous membrane and/or the first reservoir (e.g. reversibly detached). In some cases, the first reservoir is fluidically coupled to a porous membrane. In some cases, porous membrane comprises at least one nanopore, wherein the at least one nanopore is in fluid communication with the first and/or second reservoir to provide for delivery of the biomolecule through the nanopores. In some cases, the first reservoir comprises a cover. In some cases, the cover protects a sample in the first reservoir from contamination, for example, during centrifugation.

In some cases, the first reservoir has a length ranging from about 0.01 mm to about 5 mm, about 5 mm to about 10 mm, about 10 mm to about 15 mm, about 15 mm to about 20 mm, about 20 mm to about 25 mm, about 25 mm to about 30 mm, about 30 mm to about 35 mm, about 35 mm to about 40 mm, about 40 mm to about 45 mm, about 45 mm to about 50 mm, about 50 mm to about 55 mm, about 60 mm to about 65 mm, about 65 mm to about 70 mm, about 70 mm to about 75 mm, about 75 mm to about 80 mm, about 80 mm to about 95 mm, about 95 mm to about 100 mm, about 100 mm to about 105 mm, about 105 mm to about 110 mm, about 110 mm to about 115 mm, about 115 mm to about 120 mm, about 120 mm to about 125 mm, about 125 mm to about 130 mm, about 130 mm to about 135 mm, about 135 mm to about 140 mm, about 140 mm to about 145 mm, or about 145 mm to about 150 mm In some cases, the first reservoir has a width ranging from about 0.01 mm to about 5 mm, about 5 mm to about 10 mm, about 10 mm to about 15 mm, about 15 mm to about 20 mm, about 20 mm to about 25 mm, about 25 mm to about 30 mm, about 30 mm to about 35 mm, about 35 mm to about 40 mm, about 40 mm to about 45 mm, about 45 mm to about 50 mm, about 50 mm to about 55 mm, about 60 mm to about 65 mm, about 65 mm to about 70 mm, about 70 mm to about 75 mm, about 75 mm to about 80 mm, about 80 mm to about 85 mm, about 85 mm to about 90 mm, about 90 mm to about 95 mm, or about 95 mm to about 100 mm In some cases, the first reservoir has a height ranging from about 0.01 mm to about 5 mm, about 5 mm to about 10 mm, about 10 mm to about 15 mm, about 15 mm to about 20 mm, about 20 mm to about 25 mm, about 25 mm to about 30 mm, about 30 mm to about 35 mm, about 35 mm to about 40 mm, about 40 mm to about 45 mm, or about 45 mm to about 50 mm

In some cases, the first reservoir has a depth ranging from about 0.01 mm to about 10 mm In some cases, the first reservoir has a depth ranging from about 0.01 mm to about 0.1 mm, 0.1 mm to about 0.5 mm, 0.5 mm to about 1 mm, about 1 mm to about 1.5 mm, 1.5 mm to about 2 mm, 2 mm to about 2.5 mm, 2.5 mm to about 3 mm, 3 mm to about 3.5 mm, 3.5 mm to about 4 mm, 4 mm to about 4.5 mm, or 4.5 mm to about 5 mm

In some cases, the first reservoir has an area ranging from 0.5×0.5 cm² to 20×20 cm². In some cases, the first reservoir has an area ranging from 0.5×0.5 cm² to 5×5 cm², 5×5 cm² to 10×10 cm², 10×10 cm² to 15×15 cm², or 15×15 cm² to 20×20 cm².

In some cases, first reservoir is circular-shaped. In some cases, the first reservoir has a diameter ranging from about 0.01 mm to about 5 mm, about 5 mm to about 10 mm, about 10 mm to about 15 mm, about 15 mm to about 20 mm, about 20 mm to about 25 mm, about 25 mm to about 30 mm, about 30 mm to about 35 mm, about 35 mm to about 40 mm, about 40 mm to about 45 mm, about 45 mm to about 50 mm, about 50 mm to about 55 mm, about 55 mm to about 60 mm, about 60 mm to about 65 mm, about 65 mm to about 70 mm, about 70 mm to about 75 mm, about 75 mm to about 80 mm, about 80 mm to about 85 mm, about 85 mm to about 90 mm, about 90 mm to about 95 mm, or about 95 mm to about 100 mm

The first reservoir is not limited to the shapes and/or sizes as described herein and can be any shape and/or size as required per conditions specific to its intended use.

Aspects of the present disclosure include a delivery device comprising a second reservoir comprising a proximal end and a distal end. In some cases, the second reservoir is a second chamber.

In some cases, the second reservoir is formed from a cell culture dish, a cell culture plate, and/or a cell culture flask. In some cases, the second reservoir is formed from a material selected from a PS, polyethylene, polycarbonate, polyolefin, ethylene vinyl acetate, polypropylene, polysulfone, PTFE, a silicone rubber or copolymer, poly(styrene-butadiene-styrene), PDMS, polyimide, polyurethane, SU-8, PMMA, PET, PVC, PCL, or any combination thereof.

In some cases, the second reservoir is formed from a material selected from a biocompatible polymer. Biocompatible polymers include natural or synthetic polymers. Non-limiting examples of biocompatible polymers include, but are not limited to poly(alpha esters) such as poly(lactate acid), poly(glycolic acid), polyorthoesters and polyanhydrides and their copolymers, polyglycolic acid and polyglactin, cellulose ether, cellulose, cellulosic ester, fluorinated polyethylene, phenolic, poly-4-methylpentene, polyacrylonitrile, polyamide, polyamideimide, polyacrylate, polybenzoxazole, polycarbonate, polycyanoarylether, polyester, polyestercarbonate, polyether, polyetheretherketone, polyetherimide, polyetherketone, polyethersulfone, polyethylene, polyfluoroolefin, polyimide, polyolefin, polyoxadiazole, polyphenylene oxide, polyphenylene sulfide, polypropylene, polystyrene, polysulfide, polysulfone, polytetrafluoroethylene, polythioether, polytriazole, polyurethane, polyvinyl, polyvinylidene fluoride, regenerated cellulose, silicone, urea-formaldehyde, polyglactin, or copolymers or a combination of these materials. SU-8 formulations comprise a monomer, containing epoxy moieties, a solvent, and a photoacid initiator. In some cases, the solvent in the SU-8 formulation is a cyclopentanone. In some cases, the photoacid initiator in the SU-8 formulation is a triarylsulfonium hexafluoroantimonate. On exposure to UV radiation, a photoacid is produced that protonates the epoxy moieties, which then react with neutral epoxy groups on heating, resulting in a cross-linked polymer network of high mechanical strength and thermal stability. See e.g. Nemani et al. 2013, Mater Sci Eng C Mater Biol Appl. 33(7): 10.1016, which is hereby incorporated by reference in its entirety.

In some cases, the second reservoir is a cylindrical shape, a circular shape, a square shape, a spherical shape, a cylindrical shape, or a rectangular shape. In some cases, the second reservoir is sized and/or shaped to receive a sample, such as a biomolecule in a liquid medium. A second reservoir may have one or more, two or more, or three or more open ends and may include, for example, an opening for receiving fluid at a first end and/or an opening for expelling fluid at a second end. In some cases, the second reservoir includes walls that form the side boundary of the second reservoir. In some cases, the first reservoir includes walls that form the side boundary of the second reservoir. In some cases, the second reservoir is integral and/or included with a porous membrane. In some cases, the second reservoir is integral and/or included with the first reservoir. In some cases, the second reservoir is integral and/or included with the first reservoir and the porous membrane. In some cases, the second reservoir is separate e.g. reversibly disconnectable, from a porous membrane. In some cases, the second reservoir is reversibly connectable to the porous membrane and/or the first reservoir. In some cases, the second reservoir is reversibly detachable from the porous membrane. In some cases, the second reservoir is fluidically coupled and/or connected to the porous membrane. In some cases, the second reservoir is fluidically coupled and/or connected to the first reservoir. In some cases, the second reservoir is a second chamber. In some cases, the second reservoir is an electrode. In some cases, the second reservoir is a second electrode.

In some cases, the second reservoir has a length ranging from about 0.01 mm to about 5 mm, about 5 mm to about 10 mm, about 10 mm to about 15 mm, about 15 mm to about 20 mm, about 20 mm to about 25 mm, about 25 mm to about 30 mm, about 30 mm to about 35 mm, about 35 mm to about 40 mm, about 40 mm to about 45 mm, about 45 mm to about 50 mm, about 50 mm to about 55 mm, about 60 mm to about 65 mm, about 65 mm to about 70 mm, about 70 mm to about 75 mm, about 75 mm to about 80 mm, about 80 mm to about 95 mm, about 95 mm to about 100 mm, about 100 mm to about 105 mm, about 105 mm to about 110 mm, about 110 mm to about 115 mm, about 115 mm to about 120 mm, about 120 mm to about 125 mm, about 125 mm to about 130 mm, about 130 mm to about 135 mm, about 135 mm to about 140 mm, about 140 mm to about 145 mm, or about 145 mm to about 150 mm In some cases, the second reservoir has a width ranging from about 0.01 mm to about 5 mm, about 5 mm to about 10 mm, about 10 mm to about 15 mm, about 15 mm to about 20 mm, about 20 mm to about 25 mm, about 25 mm to about 30 mm, about 30 mm to about 35 mm, about 35 mm to about 40 mm, about 40 mm to about 45 mm, about 45 mm to about 50 mm, about 50 mm to about 55 mm, about 60 mm to about 65 mm, about 65 mm to about 70 mm, about 70 mm to about 75 mm, about 75 mm to about 80 mm, about 80 mm to about 85 mm, about 85 mm to about 90 mm, about 90 mm to about 95 mm, or about 95 mm to about 100 mm In some cases, the second reservoir has a height ranging from about 0.01 mm to about 5 mm, about 5 mm to about 10 mm, about 10 mm to about 15 mm, about 15 mm to about 20 mm, about 20 mm to about 25 mm, about 25 mm to about 30 mm, about 30 mm to about 35 mm, about 35 mm to about 40 mm, about 40 mm to about 45 mm, or about 45 mm to about 50 mm

In some cases, the second reservoir has a depth ranging from about 0.01 mm to about 10 mm In some cases, the second reservoir has a depth ranging from about 0.01 mm to about 0.1 mm, 0.1 mm to about 0.5 mm, 0.5 mm to about 1 mm, about 1 mm to about 1.5 mm, 1.5 mm to about 2 mm, 2 mm to about 2.5 mm, 2.5 mm to about 3 mm, 3 mm to about 3.5 mm, 3.5 mm to about 4 mm, 4 mm to about 4.5 mm, or 4.5 mm to about 5 mm

In some cases, the second reservoir has an area ranging from 0.5×0.5 cm² to 20×20 cm². In some cases, the second reservoir has an area ranging from 0.5×0.5 cm² to 5×5 cm², 5×5 cm² to 10×10 cm², 10×10 cm² to 15×15 cm², or 15×15 cm² to 20×20 cm².

In some cases, second reservoir is circular-shaped. In some cases, the second reservoir has a diameter ranging from about 0.01 mm to about 5 mm, about 5 mm to about 10 mm, about 10 mm to about 15 mm, about 15 mm to about 20 mm, about 20 mm to about 25 mm, about 25 mm to about 30 mm, about 30 mm to about 35 mm, about 35 mm to about 40 mm, about 40 mm to about 45 mm, about 45 mm to about 50 mm, about 50 mm to about 55 mm, about 55 mm to about 60 mm, about 60 mm to about 65 mm, about 65 mm to about 70 mm, about 70 mm to about 75 mm, about 75 mm to about 80 mm, about 80 mm to about 85 mm, about 85 mm to about 90 mm, about 90 mm to about 95 mm, or about 95 mm to about 100 mm

The second reservoir is not limited to the shapes and/or sizes as described herein and can be any shape and/or size as required per conditions specific to its intended use.

Aspects of the present disclosure include a delivery device comprising a porous membrane. In some cases, the porous membrane is positioned between the first reservoir and the second reservoir. In some cases, the porous membrane is positioned between the first reservoir and a second electrode, wherein the second electrode is positioned at a distal end of the porous membrane. In some cases, the porous membrane includes at least one nanopore coupled and/or connected to the first reservoir and the second reservoir. In some cases, the porous membrane includes at least one nanopore fluidically coupled to the first reservoir and the second reservoir. In some cases, the porous membrane separates the first reservoir from the second reservoir. In some cases, the porous membrane is integral with the first reservoir and/or the second reservoir. In some cases, the second reservoir is a second electrode.

In some cases, porous membrane includes at least one nanopore. In some cases, the porous membrane includes a plurality of nanopores.

In some cases, the porous membrane has an area ranging from about 1 mm² to 1000 mm². In some cases, the porous membrane has an area ranging from about 1 cm² to 500 cm². In some cases, the porous membrane has an area ranging from about 1 cm² to about 50 cm², about 50 cm² to about 100 cm², about 100 cm² to about 150 cm², about 150 cm² to about 200 cm², about 200 cm² to about 250 cm², about 250 cm² to about 300 cm², about 300 cm² to about 350 cm², about 350 cm² to about 400 cm², about 400 cm² to about 450 cm², about 450 cm² to about 500 cm², or about 500 cm² to about 550 cm².

In some cases, the porous membrane has a surface area ranging from about 1 mm² to 1000 mm². In some cases, the porous membrane has a surface area ranging from about 1 cm² to 500 cm². In some cases, the porous membrane has a surface area ranging from about 1 cm² to about 50 cm², about 50 cm² to about 100 cm², about 100 cm² to about 150 cm², about 150 cm² to about 200 cm², about 200 cm² to about 250 cm², about 250 cm² to about 300 cm², about 300 cm² to about 350 cm², about 350 cm² to about 400 cm², about 400 cm² to about 450 cm², about 450 cm² to about 500 cm², or about 500 cm² to about 550 cm².

In some cases, the porous membrane has a thickness ranging from about 1 μm to about 10 μm, about 10 μm to about 20 μm, about 20 μm to about 30 μm, or about 30 μm to about 40 μm, or about 40 μm to about 50 μm.

In some cases, the porous membrane has a length ranging from about 0.01 mm to about 5 mm, about 5 mm to about 10 mm, about 10 mm to about 15 mm, about 15 mm to about 20 mm, about 20 mm to about 25 mm, about 25 mm to about 30 mm, about 30 mm to about 35 mm, about 35 mm to about 40 mm, about 40 mm to about 45 mm, about 45 mm to about 50 mm, about 50 mm to about 55 mm, about 60 mm to about 65 mm, about 65 mm to about 70 mm, about 70 mm to about 75 mm, about 75 mm to about 80 mm, about 80 mm to about 95 mm, about 95 mm to about 100 mm, about 100 mm to about 105 mm, about 105 mm to about 110 mm, about 110 mm to about 115 mm, about 115 mm to about 120 mm, about 120 mm to about 125 mm, about 125 mm to about 130 mm, about 130 mm to about 135 mm, about 135 mm to about 140 mm, about 140 mm to about 145 mm, or about 145 mm to about 150 mm In some cases, the first reservoir has a width ranging from about 0.01 mm to about 5 mm, about 5 mm to about 10 mm, about 10 mm to about 15 mm, about 15 mm to about 20 mm, about 20 mm to about 25 mm, about 25 mm to about 30 mm, about 30 mm to about 35 mm, about 35 mm to about 40 mm, about 40 mm to about 45 mm, about 45 mm to about 50 mm, about 50 mm to about 55 mm, about 60 mm to about 65 mm, about 65 mm to about 70 mm, about 70 mm to about 75 mm, about 75 mm to about 80 mm, about 80 mm to about 85 mm, about 85 mm to about 90 mm, about 90 mm to about 95 mm, or about 95 mm to about 100 mm In some cases, the porous membrane has a height ranging from about 0.01 mm to about 5 mm, about 5 mm to about 10 mm, about 10 mm to about 15 mm, about 15 mm to about 20 mm, about 20 mm to about 25 mm, about 25 mm to about 30 mm, about 30 mm to about 35 mm, about 35 mm to about 40 mm, about 40 mm to about 45 mm, or about 45 mm to about 50 mm

In some cases, the porous membrane has a depth ranging from about 0.01 mm to about 10 mm

In some cases, the first reservoir has a depth ranging from about 0.01 mm to about 0.1 mm, 0.1 mm to about 0.5 mm, 0.5 mm to about 1 mm, about 1 mm to about 1.5 mm, 1.5 mm to about 2 mm, 2 mm to about 2.5 mm, 2.5 mm to about 3 mm, 3 mm to about 3.5 mm, 3.5 mm to about 4 mm, 4 mm to about 4.5 mm, or 4.5 mm to about 5 mm

In some cases, the porous membrane has an area ranging from 0.5×0.5 cm² to 20×20 cm². In some cases, the porous membrane has an area ranging from 0.5×0.5 cm² to 5×5 cm², 5×5 cm² to 10×10 cm², 10×10 cm² to 15×15 cm², or 15×15 cm² to 20×20 cm².

In some cases, porous membrane reservoir is circular-shaped. In some cases, the porous membrane has a diameter ranging from about 0.01 mm to about 5 mm, about 5 mm to about 10 mm, about 10 mm to about 15 mm, about 15 mm to about 20 mm, about 20 mm to about 25 mm, about 25 mm to about 30 mm, about 30 mm to about 35 mm, about 35 mm to about 40 mm, about 40 mm to about 45 mm, about 45 mm to about 50 mm, about 50 mm to about 55 mm, about 55 mm to about 60 mm, about 60 mm to about 65 mm, about 65 mm to about 70 mm, about 70 mm to about 75 mm, about 75 mm to about 80 mm, about 80 mm to about 85 mm, about 85 mm to about 90 mm, about 90 mm to about 95 mm, or about 95 mm to about 100 mm

The porous membrane is not limited to the shapes and/or sizes as described herein and can be any shape and/or size as required per conditions specific to its intended use.

In some cases, the porous membrane includes a plurality of nanopores. In some cases, the nanopore shapes include linear, square, rectangular (slit-shaped), circular, ovoid, elliptical, cylindrical, or other shapes. In some cases, the nanopore includes a single shape or a combination of shapes. As used herein, the width of the nanopore refers to the diameter where the pore is circular, cylindrical, ovoid, or elliptical. In some cases, the nanopore is cylindrical. In some cases, the sizes of the nanopores are highly uniform. In some cases, the pores are micromachined such that there is less than 20% size variability, less than 10% size variability, less than 5% size variability, less than 2% size variability, or less than 1% size variability between the dimensions of the nanopores. In some cases, the number of nanopores on the porous membrane is sufficient to allow delivery of biomolecules through the nanopores and into a eukaryotic cell. The nanopores of the porous membrane may be fabricated using any known porous membrane fabrication technique, such as a Track Etching method. The Track Etching method, described in its conventional sense, is based on the beaming of polymeric materials with energetic-heavy ions leading to the formation of linear damaged tracks across the irradiated polymeric layer or film. These tracks are then revealed into pores using known wet chemical etching techniques. The combination of the process of “tracks” with their subsequent etching is termed “Track Etching”.

In some cases, the nanopore has a pore size ranging from about 5 nm to about 150 nm. In some cases, the nanopore has a pore size ranging from about 50 nm to about 200 nm. In some cases, the nanopore has a pore size ranging from about 5 to 200 nm, e.g., about 10 nm to about 200 nm, including about 20 nm to about 100 nm, or about 30 nm to about 80 nm. In some cases, the nanopore has a pore size ranging from about 10 nm, about 20 nm, about 30 nm, about 40 nm, about 50 nm, about 60 nm, about 70 nm, about 80 nm, about 90 nm, about 100 nm, about 110 nm, about 120 nm, about 130 nm, about 140 nm, about 150 nm, about 160 nm, about 170 nm, about 180 nm, about 190 nm, or about 200 nm. In some cases, the nanopore has a pore size ranging from about 1 nm to about 10 nm, about 10 nm to about 20 nm, about 20 nm to about 30 nm, about 30 nm to about 40 nm, about 40 nm to about 50 nm, about 50 nm to about 60 nm, about 60 nm to about 70 nm, about 70 nm to about 80 nm, about 80 nm to about 90 nm, about 90 nm to about 100 nm, about 100 nm to about 110 nm, about 110 nm to about 120 nm, about 120 nm to about 130 nm, about 130 nm to about 140 nm, about 140 nm to about 150 nm, about 150 nm to about 160 nm, about 160 nm to about 170 nm, about 170 nm to about 180 nm, about 180 nm to about 190 nm, or about 190 nm to about 200 nm. In some cases, the nanopore has a pore size of from about 50 nm to about 100 nm. In some cases, the nanopore has a pore size of from about 100 nm to about 150 nm. In some cases, the nanopore has a pore size of from about 150 nm to about 200 nm.

In some cases, the size of the nanopore is smaller than the diameter of a eukaryotic cell. In some cases, a plurality of nanopores are in physical contact with a single cell. In some cases, at least about 40 nanopores, at least about 60 nanopores, at least about 80 nanopores, at least about 100 nanopores, at least about 120 nanopores, at least about 140 nanopores, at least about 160 nanopores, about 180 nanopores, or about 200 nanopores are in physical contact with a eukaryotic cell. In some cases, the number of nanopores in physical contact with a cell ranges from about 1 nanopore per cell to about 100 nanopores per cell, about 100 nanopores per cell to about 500 nanopores per cell, about 500 nanopores per cell to about 1000 nanopores per cell, about 1000 nanopores per cell to about 1500 nanopores per cell, or about 1500 nanopores per cell to about 2000 nanopores per cell. In some cases, the number of nanopores in physical contact with a cell ranges from about 10 nanopores per cell to about 20 nanopores per cell. In some cases, the number of nanopores in physical contact with a cell ranges from about 20 nanopores per cell to about 30 nanopores per cell.

In some cases, the porous membrane has a pore density ranging from about 1 nanopore per cm², about 10 nanopores per cm², about 10² nanopores per cm², about 10⁴ nanopores per cm², about 10⁵ nanopores per cm², about 10⁶ nanopores per cm², about 10⁷ nanopores per cm², about 10⁸ nanopores per cm², about 10⁹ nanopores per cm², or about 10¹⁰ nanopores per cm². In some cases, the porous membrane has a pore density ranging from about 1 nanopore per cm² to about 5×10¹⁰ nanopores per cm². In some cases, the density of nanopores on the porous membrane may be in the range of about 10⁶ to about 10¹⁰ nanopores per cm², e.g., about 1×10⁶ to about 1×10¹⁰ about 1×10⁶ to about 1×10⁹, about 1×10⁶ to about 1×10⁸, about 1×10⁶ to about 1×10⁷, about 2×10⁶ to about 2×10¹⁰, about 2×10⁶ to about 2×10⁹, about 2×10⁶ to about 2×10⁸, about 2×10⁶ to about 2×10⁷, about 3×10⁶ to about 3×10¹⁰, about 3×10⁶ to about 3×10⁹, about 3×10⁶ to about 3×10⁸, about 3×10⁶ to about 3×10⁷, about 4×10⁶ to about 4×10¹⁰, about 4×10⁶ to about 4×10⁹, about 4×10⁶ to about 4×10⁸, about 1×10⁶ to about 4×10⁷, about 5×10⁶ to about 5×10¹⁰, about 5×10⁶ to about 5×10⁹, about 5×10⁶ to about 5×10⁸, or about 5×10⁶ to about 5×10⁷. In some cases, the density of nanopores on the porous membrane may be in the range of about 10⁶ to about 10¹⁰ nanopores per cm², e.g., about 3×10⁶ to about 3×10⁸ nanopores per cm², about 10⁷ to about 3×10⁸ nanopores per cm², about 3×10⁷ to about 3×10⁸ nanopores per cm², or about 3×10⁸ to about 3×10¹⁰. In some cases, the density of nanopores on the porous membrane may be in the range of about 10⁶ to about 10¹⁰ nanopores per cm², e.g., about 4×10⁶ to about 4×10⁸ nanopores per cm², about 10⁷ to about 4×10⁸ nanopores per cm², about 4×10⁷ to about 4×10⁸ nanopores per cm², or about 4×10⁸ to 4×10¹⁰. In some cases, the density of nanopores on the porous membrane may be in the range of about 10⁶ to about 10¹⁰ nanopores per cm², e.g., about 5×10⁶ to about 5×10⁸ nanopores per cm², about 10⁷ to about 5×10⁸ nanopores per cm², about 5×10⁷ to 5×10⁸ nanopores per cm², or about 5×10⁸ to about 5×10¹⁰ nanopores per cm². In some cases, the density of nanopores on the porous membrane may be in the range of about 1×10² to about 2×10⁸ nanopores per cm², about 1×10⁸ to about 2×10¹⁰ nanopores per cm², about 2×10⁶ to about 2×10⁸ nanopores per cm², about 2×10⁴ to about 2×10⁶, or about 2×10² to about 2×10⁴. In some cases, the density of nanopores on the porous membrane may be in the range of about 1×10² to about 3×10⁸ nanopores per cm², about 1×10⁸ to about 3×10¹⁰ nanopores per cm², about 3×10⁶ to about 3×10⁸ nanopores per cm², about 3×10⁴ to about 3×10⁶, or about 3×10² to about 3×10⁴. In some cases, the density of nanopores on the porous membrane may be in the range of about 1×10² to about 4×10⁸ nanopores per cm², about 1×10⁸ to about 4×10¹⁰ nanopores per cm², about 4×10⁶ to about 4×10⁸ nanopores per cm², about 4×10⁴ to about 4×10⁶, or about 4×10² to about 4×10⁴. In some cases, the density of nanopores on the porous membrane may be in the range of about 1×10² to about 5×10⁸ nanopores per cm², about 1×10⁸ to about 5×10¹⁰ nanopores per cm², about 5×10⁶ to about 5×10⁸ nanopores per cm², about 5×10⁴ to about 5×10⁶, or about 5×10² to about 5×10⁴.

The nanopore is not limited to the shapes and/or sizes as described herein and can be any shape and/or size as required per conditions specific to its intended use.

In some cases, the porous membrane is formed from a material selected from a cell culture dish, a cell culture plate, and/or a cell culture flask. In some cases, the porous membrane is formed from a material selected from a PS, polyethylene, polycarbonate, polyolefin, ethylene vinyl acetate, polypropylene, polysulfone, PTFE, a silicone rubber or copolymer, poly(styrene-butadiene-styrene), PDMS, polyimide, polyurethane, SU-8, PMMA, PET, PVC, PCL, or any combination thereof. SU-8 formulations comprise a monomer, containing epoxy moieties, a solvent, and a photoacid initiator. In some cases, the solvent in the SU-8 formulation is a cyclopentanone. In some cases, the photoacid initiator in the SU-8 formulation is a triarylsulfonium hexafluoroantimonate. On exposure to UV radiation, a photoacid is produced that protonates the epoxy moieties, which then react with neutral epoxy groups on heating, resulting in a cross-linked polymer network of high mechanical strength and thermal stability. See e.g. Nemani et al. 2013, Mater Sci Eng C Mater Biol Appl. 33(7): 10.1016, which is hereby incorporated by reference in its entirety.

In some cases, the porous membrane is formed from a material selected from a biocompatible polymer. Biocompatible polymers include natural or synthetic polymers. Non-limiting examples of biocompatible polymers include, but are not limited to poly(alpha esters) such as poly(lactate acid), poly(glycolic acid), polyorthoesters and polyanhydrides and their copolymers, polyglycolic acid and polyglactin, cellulose ether, cellulose, cellulosic ester, fluorinated polyethylene, phenolic, poly-4-methylpentene, polyacrylonitrile, polyamide, polyamideimide, polyacrylate, polybenzoxazole, polycarbonate, polycyanoarylether, polyester, polyestercarbonate, polyether, polyetheretherketone, polyetherimide, polyetherketone, polyethersulfone, polyethylene, polyfluoroolefin, polyimide, polyolefin, polyoxadiazole, polyphenylene oxide, polyphenylene sulfide, polypropylene, polystyrene, polysulfide, polysulfone, polytetrafluoroethylene, polythioether, polytriazole, polyurethane, polyvinyl, polyvinylidene fluoride, regenerated cellulose, silicone, urea-formaldehyde, polyglactin, or copolymers or a combination of these materials.

In some cases, the delivery device has an overall area of from about 0.001 cm² to about 30 cm².

In some cases, the delivery device has an overall area of from about 0.01 cm² to about 15 cm². In some cases, the delivery device has an overall area of from about 0.01 mm² to about 15 cm². In some cases, the delivery device has an overall area of from about 0.01 mm² to about 15 cm². In some cases, the delivery device has an overall area of from 0.5×0.5 cm² to 20×20 cm². In some cases, the delivery device has an overall area of from 0.5×0.5 cm² to 5×5 cm², 5×5 cm² to 10×10 cm², 10×10 cm² to 15×15 cm², or 15×15 cm² to 20×20 cm².

In some cases, the delivery device has an overall area of from about 0.01 mm² to about 5 mm², from about 0.01 mm² to about 10 mm², from about 0.01 mm² to about 15 mm², from about 0.01 mm² to about 20 mm². In some cases, the delivery device has an overall area of from about 0.05 mm² to about 1 mm², about 0.1 mm² to about 0.5 mm², about 0.5 mm² to about 1 mm², from about 1 mm² to about 5 mm², from about 5 mm² to about 10 mm², from about 10 mm² to about 20 mm². from about 20 mm² to about 30 mm², from about 30 mm² to about 40 mm², from about 40 mm² to about 50 mm², from about 50 mm² to about 60 mm², from about 60 mm² to about 70 mm², from about 70 mm² to about 80 mm², from about 80 mm² to about 90 mm², or from about 90 mm² to about 100 mm². In some cases, the delivery device has an overall area of from about 1 mm² to about 50 mm², or from about 50 mm² to about 100 mm².

In some cases, the delivery device has an surface area of from about 0.001 cm² to about 30 cm². In some cases, the delivery device has a surface area of from about 0.01 cm² to about 15 cm². In some cases, the delivery device has a surface area of from about 0.01 mm² to about 15 cm². In some cases, the delivery device has a surface area of from about 0.01 mm² to about 15 cm². In some cases, the delivery device has a surface area of from about 0.01 mm² to about 5 mm², from about 0.01 mm² to about 10 mm², from about 0.01 mm² to about 15 mm², from about 0.01 mm² to about 20 mm². In some cases, the delivery device has a surface area of from about 0.05 mm² to about 1 mm², about 0.1 mm² to about 0.5 mm², about 0.5 mm² to about 1 mm², from about 1 mm² to about 5 mm², from about 5 mm² to about 10 mm², from about 10 mm² to about 20 mm². from about 20 mm² to about 30 mm², from about 30 mm² to about 40 mm², from about 40 mm² to about 50 mm², from about 50 mm² to about 60 mm², from about 60 mm² to about 70 mm², from about 70 mm² to about 80 mm², from about 80 mm² to about 90 mm², from about 90 mm² to about 100 mm², from about 100 mm² to about 120 mm², from about 120 mm² to about 130 mm², from about 130 mm² to about 140 mm², from about 140 mm² to about 150 mm², from about 150 mm² to about 160 mm², from about 160 mm² to about 170 mm², from about 180 mm² to about 190 mm², or from about 190 mm² to about 200 mm². In some cases, the delivery device has a surface area of from about 1 mm² to about 50 mm², from about 50 mm² to about 100 mm², from about 100 mm² to about 200 mm², from about 200 mm² to about 250 mm², or from about 250 mm² to about 300 mm².

Aspects of the present disclosure include a delivery device comprising an electrode. In some cases, the delivery device comprises at least one electrode. In some cases, the delivery device comprises at least two or more electrodes. In some cases, the delivery device comprises at least two or more, at least three or more, at least four or more, at least five or more, at least six or more, at least seven or more, at least eight or more, at least nine or more, or at least ten electrodes or more electrodes. In some cases, the delivery device includes a plurality of electrodes.

In some cases, the at least two or more electrodes have a shape or geometry that are fabricated for creating an electric field. Any suitable microfabrication, micromachining, or other known methods may be used to fabricate the at least two or more electrodes. Non-limiting examples of electrode geometries include interdigitated electrodes, circle-on-line electrodes, diamond-on-line electrodes, castellated electrodes, sinusoidal electrodes, or a combination thereof. In some cases, the electrode is a circular shape, a square shape, a spherical shape, a disk shape, an oval shape, an ellipse shape, an L-shape, a U-shape, a Z-shape, a v-shape, a tweezer shape, or a rectangular shape. In some cases, the electrode is a plate electrode or a wire electrode.

In some cases, the at least two or more electrodes are needle electrodes. In some cases, the needle electrode includes a lumen and/or channel for insertion of a syringe. In some cases, the needle electrode is a 20-gauge needle electrode, a 21-gauge needle electrode, a 22-gauge needle electrode, a 23-gauge needle electrode, a 25-gauge needle electrode, a 27-gauge needle electrode, a 30-gauge needle electrode, a 31-gauge needle electrode, or a 32-gauge needle electrode. In some cases, the at least two or more electrodes are straight tip electrodes, parallel fixed needle electrodes, chopstick electrodes, or electrodes with a bend at the tip of the electrodes. In some cases, the at least two electrodes have the same shape and/or geometry. In some cases, the at least two electrodes have a different shape and/or geometry.

In some cases, the electrodes have a length ranging from about 0.01 mm to about 5 mm, about 5 mm to about 10 mm, about 10 mm to about 15 mm, about 15 mm to about 20 mm, about 20 mm to about 25 mm, about 25 mm to about 30 mm, about 30 mm to about 35 mm, about 35 mm to about 40 mm, about 40 mm to about 45 mm, about 45 mm to about 50 mm, about 50 mm to about 55 mm, about 60 mm to about 65 mm, about 65 mm to about 70 mm, about 70 mm to about 75 mm, about 75 mm to about 80 mm, about 80 mm to about 95 mm, about 95 mm to about 100 mm, about 100 mm to about 105 mm, about 105 mm to about 110 mm, about 110 mm to about 115 mm, about 115 mm to about 120 mm, about 120 mm to about 125 mm, about 125 mm to about 130 mm, about 130 mm to about 135 mm, about 135 mm to about 140 mm, about 140 mm to about 145 mm, or about 145 mm to about 150 mm In some cases, the electrode has a width ranging from about 0.01 mm to about 5 mm, about 5 mm to about 10 mm, about 10 mm to about 15 mm, about 15 mm to about 20 mm, about 20 mm to about 25 mm, about 25 mm to about 30 mm, about 30 mm to about 35 mm, about 35 mm to about 40 mm, about 40 mm to about 45 mm, about 45 mm to about 50 mm, about 50 mm to about 55 mm, about 60 mm to about 65 mm, about 65 mm to about 70 mm, about 70 mm to about 75 mm, about 75 mm to about 80 mm, about 80 mm to about 85 mm, about 85 mm to about 90 mm, about 90 mm to about 95 mm, or about 95 mm to about 100 mm In some cases, the electrodes have a height ranging from about 0.01 mm to about 5 mm, about 5 mm to about 10 mm, about 10 mm to about 15 mm, about 15 mm to about 20 mm, about 20 mm to about 25 mm, about 25 mm to about 30 mm, about 30 mm to about 35 mm, about 35 mm to about 40 mm, about 40 mm to about 45 mm, or about 45 mm to about 50 mm

In some cases, electrodes are circular-shaped. In some cases, the electrodes have a diameter ranging from about 0.01 mm to about 5 mm, about 5 mm to about 10 mm, about 10 mm to about 15 mm, about 15 mm to about 20 mm, about 20 mm to about 25 mm, about 25 mm to about 30 mm, about 30 mm to about 35 mm, about 35 mm to about 40 mm, about 40 mm to about 45 mm, about 45 mm to about 50 mm, about 50 mm to about 55 mm, about 55 mm to about 60 mm, about 60 mm to about 65 mm, about 65 mm to about 70 mm, about 70 mm to about 75 mm, about 75 mm to about 80 mm, about 80 mm to about 85 mm, about 85 mm to about 90 mm, about 90 mm to about 95 mm, or about 95 mm to about 100 mm

In some cases, the electrodes have a width ranging from about 0.01 mm to about 5 mm, about 5 mm to about 10 mm, about 10 mm to about 15 mm, about 15 mm to about 20 mm, about 20 mm to about 25 mm, about 25 mm to about 30 mm, about 30 mm to about 35 mm, about 35 mm to about 40 mm, about 40 mm to about 50 mm In some cases, the electrodes have a width of about 1 mm, about 2 mm, about 3 mm, about 4 mm, about 5 mm, about 6 mm, about 7 mm, about 8 mm, about 9 mm, or about 10 mm In some cases, the electrodes have a height ranging from about 1 mm to about 5 mm, about 10 mm to about 15 mm, or about 15 mm to about 20 mm In some cases, the electrodes have a height of about 1 mm, about 2 mm, about 3 mm, about 4 mm, about 5 mm, about 6 mm, about 7 mm, about 8 mm, about 9 mm, or about 10 mm

In some cases, the electrode has an overall area of from about 0.001 cm² to about 30 cm². In some cases, the electrode has an overall area of from about 0.01 cm² to about 15 cm². In some cases, the electrode has an overall area of from about 0.01 mm² to about 15 cm². In some cases, the electrode has an overall area of from about 0.01 mm² to about 15 cm². In some cases, the electrode has an overall area of from about 0.01 mm² to about 5 mm², from about 0.01 mm² to about 10 mm², from about 0.01 mm² to about 15 mm², from about 0.01 mm² to about 20 mm². In some cases, the electrode has an overall area of from about 0.05 mm² to about 1 mm², about 0.1 mm² to about 0.5 mm², about 0.5 mm² to about 1 mm², from about 1 mm² to about 5 mm², from about 5 mm² to about 10 mm², from about 10 mm² to about 20 mm². from about 20 mm² to about 30 mm², from about 30 mm² to about 40 mm², from about 40 mm² to about 50 mm², from about 50 mm² to about 60 mm², from about 60 mm² to about 70 mm², from about 70 mm² to about 80 mm², from about 80 mm² to about 90 mm², or from about 90 mm² to about 100 mm². In some cases, the electrode has an overall area of from about 1 mm² to about 50 mm², or from about 50 mm² to about 100 mm².

In some cases, the active surface of the electrode has a surface area of from 0.5×0.5 cm² to 20×20 cm². In some cases, the active surface of the electrode has a surface area of from 0.5×0.5 cm²to 5×5 cm², 5×5 cm² to 10×10 cm², 10×10 cm² to 15×15 cm², or 15×15 cm² to 20×20 cm².

The electrode is not limited to the shapes and/or sizes as described herein and can be any shape and/or size as required per conditions specific to its intended use.

In some cases, the delivery device further comprises a control device for controlling the electric field produced by the at least two or more electrodes. In some cases, the control device is an electrical pulse generator. In some cases, the at least two electrodes are in connection with the electrical pulse generator.

In some cases, positioning and placement of the electrodes generates an electric field to the first and/or second reservoir, thereby introducing the biomolecules into the cells (Mir, L. M., Therapeutic perspectives of in vivo cell electropermeabilization. Bioelectrochemistry, 2001. 53:p. 1-10). In some cases, positioning and placement of the electrodes generates an electric field between the first reservoir and the second reservoir, thereby introducing the biomolecules into the cells. In some cases, the electric field is applied to the second reservoir. In some cases, the electric field is applied to the first reservoir. In some cases, the electric field is applied from the second reservoir to the first reservoir. In some cases, the electric field is applied from the first reservoir to the second reservoir. In some cases, the electric field is applied between the first reservoir and the second reservoir. In some cases, the electric field provides for permeabilization of the cell membrane. In some cases, permeabilization of the cell membrane can be reversible, e.g. temporarily permeable. In some cases, the cell membrane will reseal after a period time, such as, when the electric pulses cease. In some cases, a first electrode of the at least two electrodes is configured for insertion at the distal end of the first reservoir of the delivery device. In some cases, a second electrode of the at least one electrode is configured for insertion at the distal end of the second reservoir of the delivery device. In some cases, the first electrode is inserted and/or positioned from above into or around the distal end of the first reservoir and the second electrode is inserted and/or positioned from below into or around the distal end of the second reservoir. In some cases, the first electrode is positioned at the distal end of the first reservoir. In some cases, the second electrode is positioned at the distal end of the second reservoir. In some cases, the first and/or second electrode is in the plane of the first and/or second reservoir. In some cases, the first and/or second electrode is outside the plane of the first and/or second reservoir.

In some cases, the at least two electrodes can be electrically connected to a power source. In some cases, the delivery device includes a power source and electrical connections from the power source to the at least two electrodes. In some cases, the electrodes can be electrically connected to a power source for the administration of electrical pulses. In some cases, the power source provides electrical pulses to the electrodes for durations, voltages, current amounts, and combinations thereof to apply an electric field to the cells within the delivery device. In some cases, the electric field is applied to the first reservoir of the delivery device. In some cases, the electric field is applied to the second reservoir of the delivery device. In some cases, the electric field is applied from the first reservoir to the second reservoir of the delivery device.

In some cases, the electric field comprises a voltage ranging from 5 volts to 100 volts. In some cases, the electric field comprises a voltage ranging from 15 volts to 80 volts. In some cases, the electric field comprises a voltage ranging from 30 volts to 80 volts. In some cases, the electric field comprises a voltage ranging from 50 volts to 80 volts. In some cases, the electric field comprises a voltage ranging from 5 volts to 10 volts, 10 volts to 15 volts, 15 volts to 20 volts, 20 volts to 30 volts, 30 volts to 35 volts, 35 volts to 40 volts, 40 volts to 45 volts, 45 volts to 50 volts, 50 volts to 55 volts, 55 volts to 60 volts, 60 volts to 65 volts, 65 volts to 70 volts, 70 volts to 75 volts, 75 volts to 80 volts, 80 volts to 85 volts, 85 volts to 90 volts, 90 volts to 95 volts, or 95 volts to 100 volts. In some cases, the electric field comprises a voltage of 30 volts.

In some cases, the pulse generator is configured to generate a frequency ranging from about 1 Hz to about 1 MHz. In some cases, the pulse generator is configured to generate a frequency ranging from about 1 Hz to about 1000 Hz. In some cases, the pulse generator is configured to generate a frequency ranging from 1 Hz to 100 Hz. In some cases, the pulse generator is configured to generate a frequency ranging from 1 Hz to 25 Hz, from 25 Hz to 50 Hz, or from 50 Hz to 100 Hz. In some cases, the pulse generator is configured to generate a frequency ranging from 1 Hz to 10 Hz, from 10 Hz to 20 Hz, or from 20 Hz to 30 Hz, from 30 Hz to 40 Hz, or from 40 Hz to 50 Hz.

In some cases, the duration of the electric pulses may include nanosecond pulses, microsecond pulses, or millisecond pulses. In some cases, the duration of the electric pulses may include a pulse duration ranging from 1 microsecond to 10 milliseconds. In some cases, the duration of the electric pulses may include a pulse duration ranging from 1 to 5 milliseconds. In some cases, the duration of the electric pulses may include a pulse duration ranging from 0.001 milliseconds to 2 milliseconds. In some cases, the duration of the electric pulses may include a pulse duration ranging from 1 microsecond to 2000 microseconds. In some cases, the duration of the electric pulses may include a pulse duration ranging from 200 microseconds to 2000 microseconds. In some cases, the duration of the electric pulses may include a pulse duration ranging from 100 to 500 microseconds, 500 to 1000 microseconds, 1000 to 1500 microseconds, or from 1500 to 2000 microseconds.

In some cases, the electrode may include any conductive material, including but not limited to, titanium, gold, silver, tin oxide, indium tin oxide (ITO), or platinum.

### Delivery Methods

Aspects of the present disclosure include a method of delivering a biomolecule into a eukaryotic cell. The method comprises applying an electric field to liquid present in a delivery device of the present disclosure. Application of the electric field provides for delivery of the biomolecule into the eukaryotic cell.

An electric field is applied across the porous membrane, which results in 1) opening of the cell membrane (co-localized with the nanopores) of cells induced by the electric field across the nanopores of the porous membrane, and 2) migration of the biomolecules through the nanopores under the influence of the electric field applied to the porous membrane (i.e. electrophoretic movement), such that the biomolecule(s) enter the cell(s).

In some cases, the delivery device includes a first reservoir for culturing at least one eukaryotic cell. In some cases, the first reservoir of the delivery device of the present disclosure includes a eukaryotic cell. In some cases, the first reservoir of the delivery device of the present disclosure includes a plurality of eukaryotic cells. In some cases, the eukaryotic cell is present in a liquid medium in the first reservoir and is in physical contact with the porous membrane.

In some cases, the device includes a first reservoir for culturing a plurality of eukaryotic cells. In some cases, the device includes a first reservoir for culturing 2 or more, 10 or more, 100 or more, 1,000 or more, 5,000 or more, 10⁴ or more, 10⁵ or more, 10⁶ or more, 10⁷ or more, 10⁸ or more, 10⁹ or more, or 10¹⁰ or more cells.

In some cases, the cell is a mammalian cell. Non-limiting examples of cells include a rodent cell, a human cell, a non-human primate cell, etc. Any type of cell may be of interest (e.g. a stem cell, e.g. an embryonic stem (ES) cell, an induced pluripotent stem (iPS) cell, a germ cell; a somatic cell, e.g. a fibroblast, a hematopoietic cell, a neuron, a muscle cell, a bone cell, a hepatocyte, a pancreatic cell; an in vitro or in vivo embryonic cell of an embryo at any stage, e.g., a 1-cell, 2-cell, 4-cell, 8-cell, etc. stage zebrafish embryo; etc.). Cells may be from established cell lines or they may be primary cells, where “primary cells”, “primary cell lines”, and “primary cultures” are used interchangeably herein to refer to cells and cells cultures that have been derived from a subject and allowed to grow in vitro for a limited number of passages, i.e. splittings, of the culture. For example, primary cultures include cultures that may have been passaged 0 times, 1 time, 2 times, 4 times, 5 times, 10 times, or 15 times, but not enough times go through the crisis stage. Primary cell lines can be maintained for fewer than 10 passages in vitro.

In some cases, the cell is selected from the group consisting of: a eukaryotic cell, a eukaryotic single-cell organism, a somatic cell, a germ cell, a stem cell, a plant cell, an algal cell, an animal cell, in invertebrate cell, a vertebrate cell, a fish cell, a frog cell, a bird cell, a mammalian cell, a pig cell, a cow cell, a goat cell, a sheep cell, a rodent cell, a rat cell, a mouse cell, a non-human primate cell, a human cell, and a combination thereof.

In some cases, the biomolecule is present in a liquid medium in the second reservoir. In some cases, the liquid medium is a cell culture medium. In some cases, the liquid medium is an extracellular buffer. In some cases, the extracellular buffer comprises NaCl, KCl, HEPES, CaCl₂, MgCl₂, MgSO₄, glycerol, glucose, TCEP (tris(2-carboxyethyl)phosphine, phosphate buffer solution (PBS), water, tris buffers with different pH ranges, or a combination thereof. In some cases, the liquid medium is a combination of a buffer and a cell culture medium.

In some cases, the biomolecules are injected into the second reservoir through an opening of the second reservoir. In some cases, the volume of the biomolecules injected into the reservoir ranges from 1 μl to 1 ml. In some cases, the volume of the biomolecules injected into the reservoir ranges from 1 μl to 5 μl. In some cases, the biomolecules are injected into the reservoir using a syringe. In some cases, the diameter of the opening is of from 0.001 mm to 1 mm In some cases, the diameter of the opening is 1 mm

In some cases, the second reservoir is the second electrode. In such cases, the biomolecules are deposited on the top surface (i.e. proximal end of the second electrode) of the second electrode in the form of a liquid droplet. In such cases, the porous membrane of the delivery device is placed on top of the liquid droplet deposited on the top surface (e.g. proximal end) of the second electrode. In some cases, the porous membrane that is integral with the first reservoir of the delivery device is placed on top of the liquid droplet deposited on the top surface of the second electrode. In some cases, the volume of the liquid droplet containing the biomolecules ranges from 1 μl to 5 μl.

In some cases, the first reservoir includes a population of eukaryotic cells, and wherein a biomolecule is delivered into at least 50% of the population of eukaryotic cells. In some cases, at least 50% of the population of eukaryotic cells remains viable following application of an electrical field. In some cases, the population of eukaryotic cells is a population of mammalian cell lines.

In some cases, the second reservoir includes one or more biomolecules. In some cases, the second reservoir includes a plurality of biomolecules. In some cases, the biomolecule is a nucleic acid, a polypeptide, or a combination thereof. In some cases, the biomolecule is a deoxyribonucleic acid (DNA), a ribonucleic acid (RNA), a protein, a ribonucleoprotein (RNP), or a deoxyribonucleoprotein (DNP). Non-limiting examples of biomolecules include salts and molecular ions in solution, small molecules, proteins, genetic material (e.g. DNA, RNA, small interfering RNA (siRNA), micro RNA (miRNA), single-guide RNA (sgRNA)), synthetic constructs and nanoparticles, combinations thereof, and the like. In some cases, the biomolecule is a complementary DNA (cDNA) from eukaryotic messenger RNA (mRNA), a genomic DNA sequence from eukaryotic DNA, a synthetic nucleic acid, or a combination thereof. In some cases, the RNA comprises a single-molecule CRISPR (cluster regularly interspaced short palindromic repeats)/Cas effector polypeptide guide RNA. In some cases, the RNP comprises a CRISPR/Cas effector polypeptide and a guide RNA.

In some cases, the method comprises reversibly attaching the second reservoir onto the porous membrane. In some cases, the method comprises reversibly detaching the second reservoir onto the porous membrane. In some cases, the method comprises slidably attaching or detaching the second reservoir onto the porous membrane. In some cases, the method comprises injecting and/or transporting the biomolecules in a liquid medium into the second reservoir before applying the electric field. In some cases, the method comprises attaching the second reservoir comprising the biomolecules in a liquid medium onto the porous membrane before applying an electric field.

In some cases, the method comprises centrifuging a eukaryotic cell present in the first reservoir of the delivery device before applying the electric field. In some cases, centrifuging a eukaryotic cell before applying the electric field provides for physical contact and/or adherence of the cell to the porous membrane. In some cases, centrifuging comprises centrifuging eukaryotic cells suspended in a liquid medium to provide for physical contact of the suspended cell to the porous membrane. In some cases, the eukaryotic cell stretches and/or spreads across a plurality of nanopores when the eukaryotic cell is cultured in the delivery device.

In some cases, centrifuging a eukaryotic cell present in the first reservoir comprises centrifuging the delivery device before applying the electric field. In some cases, second reservoir is detached from the delivery device before centrifuging the eukaryotic cell. In some cases, the method comprises centrifuging a population eukaryotic cells by placing the first reservoir and the porous membrane in a well of a cell culture plate and centrifuging the population of eukaryotic cells in a centrifuge at 150 g. In some cases, the method comprises placing a cover on the first reservoir before centrifuging the eukaryotic cell. In some cases, the method comprises centrifuging a population eukaryotic cells by placing the first reservoir, the second reservoir, and the porous membrane in a well of a cell culture plate and centrifuging the population of eukaryotic cells in a centrifuge at 150 g. In some cases, the cell culture plate is a standard 6-well, 12-well, or 24-well cell culture plate.

In some cases, the eukaryotic cells are centrifuged for at least 1 minute, at least 2 minutes, at least 3 minutes, at least 4 minutes, at least 5 minutes, at least 6 minutes, at least 7 minutes, at least 8 minutes, at least 9 minutes, or at least 10 minutes.

In some cases, the cells are centrifuged at a centrifugal force ranging from 100 g to 150 g, 150 g to 200 g, 200 g to 250 g, 250 g to 300 g, 300 to 350 g, 350 g to 400 g, 400 g to 450 g, 450 g to 500 g, 500 g to 550 g, 550 g to 600 g, 600 g to 650 g, 650 g to 700 g, 700 g to 800 g, 800 g to 850 g, 850 g to 900 g, 900 g to 1000 g.

In some cases, the method comprises culturing the eukaryotic cell present in the first reservoir after the eukaryotic cell is centrifuged and before applying the electric field. In some cases, the eukaryotic cell is cultured in a liquid medium overnight. In some cases, the liquid medium is a cell culture medium. In some cases, the method comprises culturing the eukaryotic cell present in the first reservoir for a period of time to allow the eukaryotic cell to be in physical contact with the porous membrane before applying the electric field. In some cases, the period of time ranges from about 8 hours to about 10 hours, from about 10 hours to about 12 hours, from about 12 hours to about 14 hours, or from about 14 hours to about 16 hours. In some cases, the eukaryotic cell is cultured in a liquid medium overnight to adhere to the surface of porous membrane. In some cases, the eukaryotic cell is cultured in a liquid medium for about 5 minutes, about 6 minutes, about 7 minutes, about 8 minutes, about 9 minutes, or about 10 minutes. In some cases, the eukaryotic cell is cultured in a liquid medium for about 10 minutes. In some cases, the eukaryotic cell is cultured to provide a population of eukaryotic cells in the first reservoir and/or porous membrane. Any suitable cell culture medium may be used to culture the cells. Non-limiting examples of cell culture medium include Dulbecco's Modified Eagle Medium (DMEM), DMEM with Nutrient Mixture F-12 (DMEM/F12), F10 Nutrient Mixture, Media 199, Minimum Essential Media (MEM), RPMI medium, Opti-Mem I reduced Serum Media, Iscove's Modified Dulbecco's Medium (IMDM), neurobasal plus medium, a combination thereof, and the like. In some cases, the eukaryotic cell is cultured in PBS or an electroporation buffer.

In some cases, the liquid medium in the first and/or second reservoir is a buffer. In some cases, the buffer comprises NaCl, KCl, HEPES, CaCl₂, MgCl₂, MgSO₄, glycerol, glucose, TCEP (tris(2-carboxyethyl)phosphine, water, PBS, tris buffer with different pH ranges, or a combination thereof. In some cases, the liquid medium is a combination of a buffer and a cell culture medium.

In some cases, positioning and placement of the electrodes generates an electric field to the first and/or second reservoir, thereby introducing the biomolecules into the cells (Mir, L. M., Therapeutic perspectives of in vivo cell electropermeabilization. Bioelectrochemistry, 2001. 53:p. 1-10). In some cases, the method comprises applying an electric field to the second reservoir. In some cases, the method comprises applying an electric field to the first reservoir. In some cases, the method comprises applying the electric field from the second reservoir to the first reservoir. In some cases, the method comprises applying the electric field from the first reservoir to the second reservoir. In some cases, the electric field provides for permeabilization of the cell membrane. In some cases, permeabilization of the cell membrane can be reversible, e.g. temporarily permeable. In some cases, the cell membrane will reseal after a period time, such as, when the electric pulses cease.

In some cases, the method comprises inserting a first electrode of the at least two electrodes at the distal end of the first reservoir of the delivery device. In some cases, the method comprises inserting a second electrode of the at least one electrodes at the distal end of the second reservoir of the delivery device. In some cases, the method comprises inserting and/or positioning, from above, the first electrode into, or around the distal end of the first reservoir; and/or inserting and/or positioning the second electrode, from below, the second electrode into or around the distal end of the second reservoir. In some cases, the method comprises positioning the first electrode at the distal end of the first reservoir. In some cases, the method comprises positioning the second electrode at the distal end of the second reservoir. In some cases, the first and/or second electrode is in the plane of the first and/or second reservoir. In some cases, the first and/or second electrode is outside the plane of the first and/or second reservoir.

In some cases, the method comprises electrically connecting the at least two electrodes to a power source. In some cases, the delivery device includes a power source and electrical connections from the power source to the at least two electrodes. In some cases, the electrodes can be electrically connected to a power source for the administration of electrical pulses. In some cases, the power source provides electrical pulses to the electrodes for durations, voltages, current amounts, and combinations thereof to apply an electric field to the cells within the delivery device. In some cases, the method comprises applying the electric field to the first reservoir of the delivery device. In some cases, the method comprises applying the electric field to the second reservoir of the delivery device. In some cases, the method comprises applying the electric field from the first reservoir to the second reservoir of the delivery device.

In some cases, the electric field comprises a voltage ranging from 5 volts to 100 volts. In some cases, the electric field comprises a voltage ranging from 15 volts to 80 volts. In some cases, the electric field comprises a voltage ranging from 30 volts to 80 volts. In some cases, the electric field comprises a voltage ranging from 50 volts to 80 volts. In some cases, the electric field comprises a voltage ranging from 5 volts to 10 volts, 10 volts to 15 volts, 15 volts to 20 volts, 20 volts to 30 volts, 30 volts to 35 volts, 35 volts to 40 volts, 40 volts to 45 volts, 45 volts to 50 volts, 50 volts to 55 volts, 55 volts to 60 volts 60 volts to 65 volts, 65 volts to 70 volts 70 volts to 75 volts, 75 volts to 80 volts, 80 volts to 85 volts, 85 volts to 90 volts, 90 volts to 95 volts, or 95 volts to 100 volts. In some cases, the electric field comprises a voltage of 30 volts.

In some cases, the method comprises generating a frequency ranging from about 1 Hz to about 1 MHz. In some cases, the frequency is generated with a pulse generator. In some cases, the pulse generator is configured to generate a frequency ranging from about 1 Hz to about 1 MHz. In some cases, the pulse generator is configured to generate a frequency ranging from 1 Hz to 100 Hz. In some cases, the pulse generator is configured to generate a frequency ranging from 1 Hz to 25 Hz, from 25 Hz to 50 Hz, or from 50 Hz to 100 Hz. In some cases, the pulse generator is configured to generate a frequency ranging from 1 Hz to 10 Hz, from 10 Hz to 20 Hz, or from 20 Hz to 30 Hz, from 30 Hz to 40 Hz, or from 40 Hz to 50 Hz.

In some cases, the duration of the electric pulses may include nanosecond pulses, microsecond pulses, or millisecond pulses. In some cases, the duration of the electric pulses may include a pulse duration ranging from 1 microsecond to 10 milliseconds. In some cases, the duration of the electric pulses may include a pulse duration ranging from 1 to 5 milliseconds. In some cases, the duration of the electric pulses may include a pulse duration ranging from 0.001 milliseconds to 2 milliseconds. In some cases, the duration of the electric pulses may include a pulse duration ranging from 200 microseconds to 2000 microseconds. In some cases, the duration of the electric pulses may include a pulse duration ranging from 100 to 500 microseconds, 500 to 1000 microseconds, 1000 to 1500 microseconds, or from 1500 to 2000 microseconds.

In some cases, the method comprises delivering a biomolecule into at least 50% of a population of eukaryotic cells. In some cases, the method comprises delivering a biomolecule into at least 50%, at least 60%, at least 70%, at least 80%, at least 90%, or at least 100% of a population of eukaryotic cells. In some cases, the method comprises delivering the biomolecule into at least 50%, at least 55%, at least 60%, at least 65%, at least 70%, at least 75%, at least 80%, at least 85%, at least 90%, at least 95%, or at least 100% of the population of eukaryotic cells.

In some cases, at least 50% of the population of the eukaryotic cells remains viable following application of the electric field. In some cases, at least 50%, at least 60%, at least 70%, at least 80%, at least 90%, or at least 100% of the population of the eukaryotic cells remains viable following application of an electric field. In some cases, at least 50%, at least 55%, at least 60%, at least 65%, at least 70%, at least 75%, at least 80%, at least 85%, at least 90%, at least 95%, or at least 100% of the population of the eukaryotic cells remains viable following application of an electric field.

In some cases, the method further comprises assessing vitality and/or viability of the eukaryotic cell. In some cases, vitality and/or viability of the eukaryotic cell is assessed before use of the delivery device. In some cases, vitality and/or viability of the eukaryotic cell is assessed after use of the delivery device. In some cases, vitality and/or viability of the eukaryotic cell is assessed before use and after use of the delivery device. Assessing cell viability and/or cell vitality may be measured by one of many indicators of cell viability and/or cell vitality, including intracellular esterase activity, plasma membrane integrity, metabolic activity, gene expression, and protein expression. In some cases, cell vitality may be assessed by measuring glucose metabolism, calcium ion transport, ATP production, pH level, lactate formation, redox state, electromotive potential, and/or oxygen consumption of the cell.

In some cases, cell viability may be assessed by use of a label, such as a dye or a stain, that cannot pass the intact membrane of a live cell, but which enters the cytoplasm and nucleus of dead cells. Non-limiting examples of such molecules include propidium iodide and ethidium monoazide, which intercalate or covalently bind to DNA.

In some cases, the label is a fluorescent dye or a luminescent dye. A fluorescent dye may be a fluorescent polypeptide (e.g., cyan fluorescent protein (CFP), green fluorescent protein (GFP) or yellow fluorescent protein (YFP), red fluorescent protein (RFP), mCherry, etc.), a small-molecule dye (e.g., a Cy dye (e.g., Cy3, Cy5, Cy5.5, Cy 7), an Alexa dye (e.g., Alexa Fluor 488, Alexa Fluor 546, Alexa Fluor 647, Alexa Fluor 680, Alexa Fluor 750), a Visen dye (e.g. VivoTag680, VivoTag750), an S dye (e.g., S0387), a DyLight fluorophore (e.g., DyLight 750, DyLight 800), an IRDye (e.g., IRDye 680, IRDye 800), a fluorescein dye (e.g., fluorescein, carboxyfluorescein, fluorescein isothiocyanate (FITC)), a rhodamine dye (e.g., rhodamine, tetramethylrhodamine (TAMRA)) or a HOECHST dye) or a quantum dot. One or more dye(s) may be combined.

In some cases, cell viability is assessed using a LIVE/DEAD® Viability/Cytotoxicity Assay kit, which includes a fluorescent cell-permeable dye calcein AM which is retained within live cells, and ethidium homodimer (EthD-1) that enters cells with damaged membranes. As a result, live and dead cells can be easily distinguished based on the fluorescence intensity of the fluorophore used for the viability stain.

In some cases, cell viability, cell vitality and/or cell density of may be assessed using a cellometer and a membrane impermeable dye. In some cases, cell viability, cell vitality, and/or cell density may be assessed using a hemocytometer and a membrane impermeable dye. In some cases, the cells viability, cell vitality, and/or cell density can be assessed by flow cytometry, or by using a plate reader device. In some cases, cell viability, cell vitality, and/or cell density may be assessed using any microscopy method.

In some cases, cell viability is assessed by using fluorescently labeled affinity binders specific for cell death markers, such as cleaved Caspase 3, cleaved Parp or Annexin V.

In some cases, cell viability is assessed by any immunological method (e.g., enzyme-lined immunosorbent assay (ELISA), flow cytometry, Western Blot, fluorescence correlation spectroscopy (FCS), and/or fluorescence cross-correlation spectroscopy (FCCS)).

In some cases, assessing cell viability comprises labeling the eukaryotic cell with a radioactive label, a spin label, a fluorescent label or a luminescent label. The label may be conjugated to the eukaryotic cell directly or via a functional linker, (e.g., a peptide linker, a polyethylene glycol (PEG) linker, a saccharide linker, a fatty acid linker, an alkyl linker, etc.). Alternatively, one or more labeled antibody/antibodies or derivatives thereof may be labeled and bound to the eukaryotic cell. Non-limiting examples of labels used to assess cell viability can be found in U.S. Pat. No. 9,994,854, which is hereby incorporated by reference in its entirety.

In some cases, assessing cell viability comprises detecting fluorescence emitted from the labeled eukaryotic cell. In such cases, fluorescence may be detected using known microscopy methods. Non-limiting examples of microscopic methods that may be used to assess cell viability include fluorescent light microscopy, confocal microscopy, fluorescent molecular tomography (FMT), fluorescence molecular imaging (FMI), bright-field microscopy, FCS, FCCS, or fluorescence depolarization. Non-limiting examples of microscopic methods used to assess cell viability can be found in U.S. Pat. No. 9,994,854, which is hereby incorporated by reference in its entirety.

### Examples of Non-Limiting Aspects of the Disclosure

Aspects, including cases, of the present subject matter described above may be beneficial alone or in combination, with one or more other aspects or cases. Without limiting the foregoing description, certain non-limiting aspects of the disclosure numbered 1-22 are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below:

Aspect 1. A delivery device for delivering a biomolecule into a eukaryotic cell, the device comprising: a first reservoir comprising a proximal end and a distal end; a second reservoir comprising a proximal end and a distal end; a porous membrane comprising at least one nanopore with a pore size ranging from about 50 nm to about 150 nm, wherein the at least one nanopore is fluidically connected to the first reservoir and the second reservoir; and two or more electrodes configured to generate an electric field across a porous membrane.

Aspect 2. The device of claim 1, wherein the at least one nanopore has a pore size of from 50 nm to about 100 nm.

Aspect 3. The device of Aspect 2, wherein the at least one nanopore has a pore size of from 100 nm to about 150 nm.

Aspect 4. The device of Aspect 1, wherein the porous membrane comprises a nanopore density ranging from 1×10⁸ nanopores per cm² to 5×10⁸ nanopores per cm².

Aspect 5. The device of Aspect 1, wherein the porous membrane comprises a polymer material.

Aspect 6. The device of Aspect 1, wherein the porous membrane comprises an elastomer, a thermoset, a thermoplastic, glass, quartz, or a silicon material.

Aspect 7. The device of Aspect 5, wherein the material comprises polydimethylsiloxane (PDMS)), polyimide, polyurethane, SU-8, polymethylmethacrylate (PMMA), polycarbonate (PC), polystyrene (PS), polyethylene terephthalate (PET), polyvinylchloride (PVC)), or polycaprolactone (PCL).

Aspect 8. The device of Aspect 1, wherein the two or more electrodes comprise a first electrode and a second electrode.

Aspect 9. The device of Aspect 8, wherein the first electrode is positioned at the distal end of the first reservoir and the second electrode is positioned at the distal end of the second reservoir.

Aspect 10. The device of Aspect 1, wherein the device has an overall area of from about 0.01 cm² to about 15 cm².

Aspect 11. The device of any one of Aspects 1-13, wherein the thickness of the porous membrane ranges from 10 μm to 100 μm.

Aspect 12. The device of any one of claims 1-14, wherein the two or more electrodes are two or more platinum or titanium electrodes.

Aspect 13. A method of delivering a biomolecule into a eukaryotic cell, the method comprising: applying an electric field across a porous membrane of the delivery device of any one of Aspects 1-13, wherein the biomolecule is present in a liquid medium in the second reservoir, wherein the eukaryotic cell is present in a liquid medium in the first reservoir and is in physical contact with the porous membrane, and wherein application of the electric field provides for delivery of the biomolecule into the eukaryotic cell.

Aspect 14. The method of Aspect 14, further comprising centrifuging the eukaryotic cell present in the first reservoir of the delivery device before applying the electric field.

Aspect 15. The method of Aspect 15, further comprises culturing the at least one eukaryotic cell at a proximal end of the first reservoir for a period of time to allow the at least one eukaryotic cell to contact the porous membrane.

Aspect 16. The method of any one of Aspects 14-16, wherein the electric field comprises a voltage ranging from 15 volts to 80 volts.

Aspect 17. The method of Aspect 17, wherein the electric field comprises a voltage ranging from 50 volts to 80 volts.

Aspect 18. The method of any one of Aspects 14-18, wherein the biomolecule is selected from the group consisting of a DNA, an RNA, a polypeptide, ribonucleoprotein (RNP), and a deoxyribonucleoprotein (DNP), and combinations thereof.

Aspect 19. The method of Aspect 19, wherein the RNA is a single-molecule CRISPR/Cas effector peptide guide RNA.

Aspect 20. The method of Aspect 19, wherein the RNP comprises a CRISPR/Cas effector polypeptide and a guide RNA.

Aspect 21. The method of any one of Aspects 14-21, wherein the first reservoir comprises a population of eukaryotic cells, and wherein the biomolecule is delivered into at least 50% of the population of eukaryotic cells.

Aspect 22. The method of any one of Aspects 14-22, wherein at least 50% of the population of eukaryotic cells remains viable following application of the electric field.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or sec, second(s); min, minute(s); h or hr, hour(s); aa, amino acid(s); kb, kilobase(s); bp, base pair(s); nt, nucleotide(s); i.m., intramuscular(ly); i.p., intraperitoneal(ly); s.c., subcutaneous(ly); and the like.

### Example 1: Delivery Device for Delivering a Biomolecule into a Eukaryotic Cell

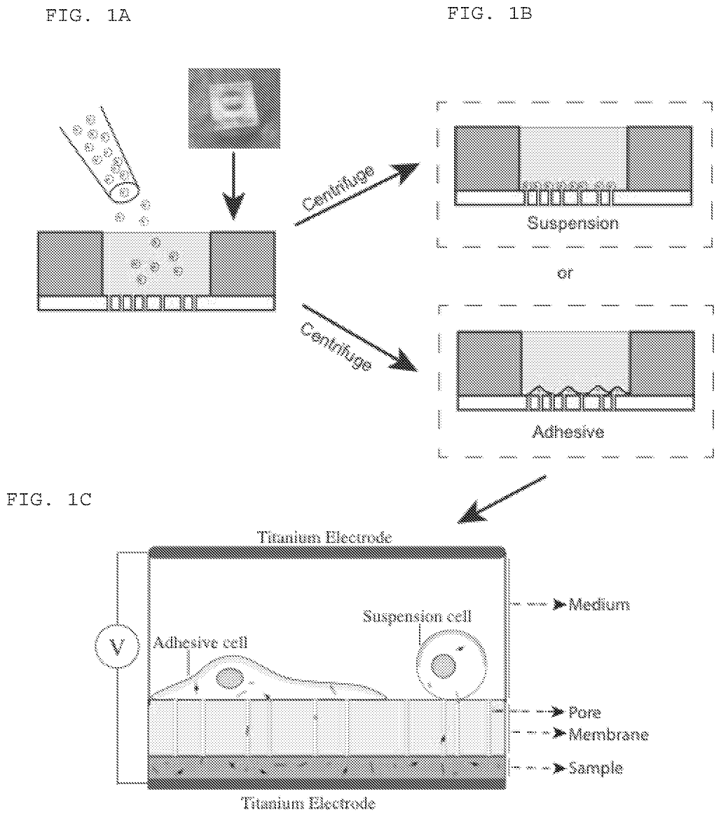

The delivery device and method of the present disclosure includes a non-toxic universal delivery device that simplifies intracellular transfection for all cell types. The delivery device includes a porous membrane as a medium to deliver biomolecules into cells, as illustrated in FIGS. 1A-1C and FIG. 10. Cells were placed in a first reservoir (e.g. cell culture reservoir) that included a holder bottom-sealed with a polycarbonate nanoporous membrane (FIG. 1A). To achieve the optimal delivery capacity, adhesive cells were allowed to spread out (i.e. extend) on the reservoir overnight before delivery, as routinely performed in cell splitting/passaging processes (FIG. 1B). However, overnight culturing was not necessary for cells in suspension. Gentle centrifugation was performed to provide for physical contact to the nanoporous membrane before applying an electric field to the cells (FIG. 1B). Biomolecules including nucleic acids, proteins, small signaling molecules, or RNP complexes, were electrophoretically dragged into the cells through the nanopores of the membrane below the cells by applying low intensity electric pulses (FIG. 1C). Since cell membrane openings induced by the applied electric field through the nanopores were transient, ultra-small, and co-localized with nanopores, this process caused little to no detectable damages to the cells. Assessing for damages to the cells included measuring the leakage of lactate dehydrogenase (LDH) in the culture media and the analyzing the expression profile of DNA damage inducible transcript 3 gene (DDIT3) in transfected cells.

Highly Efficient Transfection of Nucleic Acids into Adhered Cells

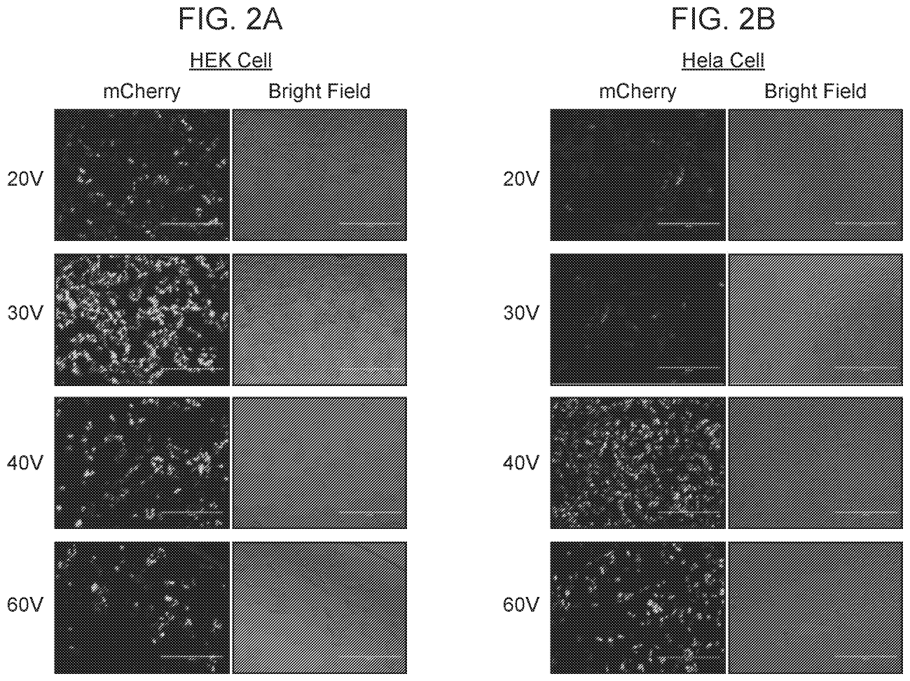

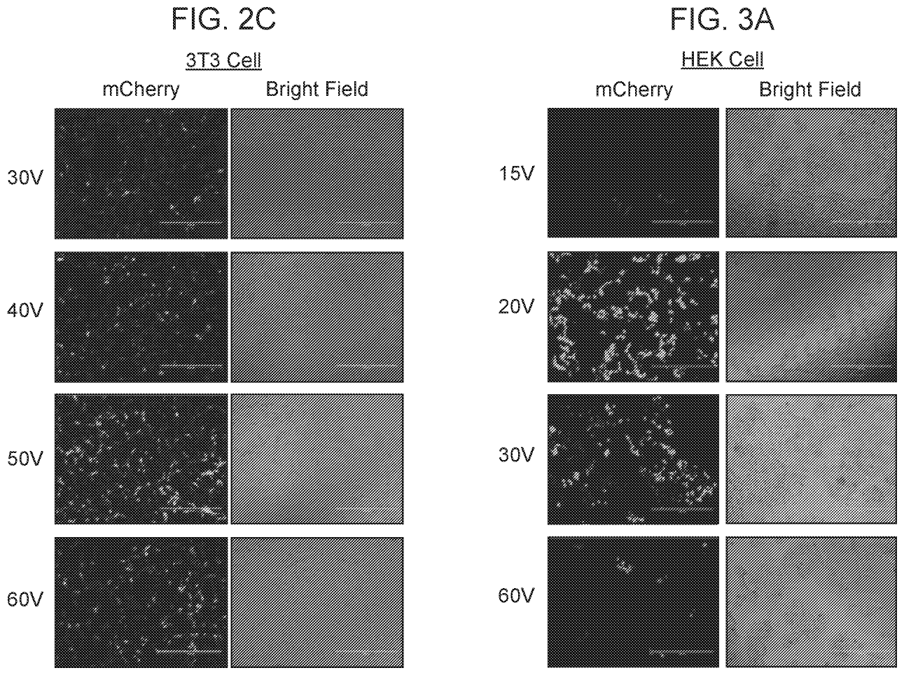

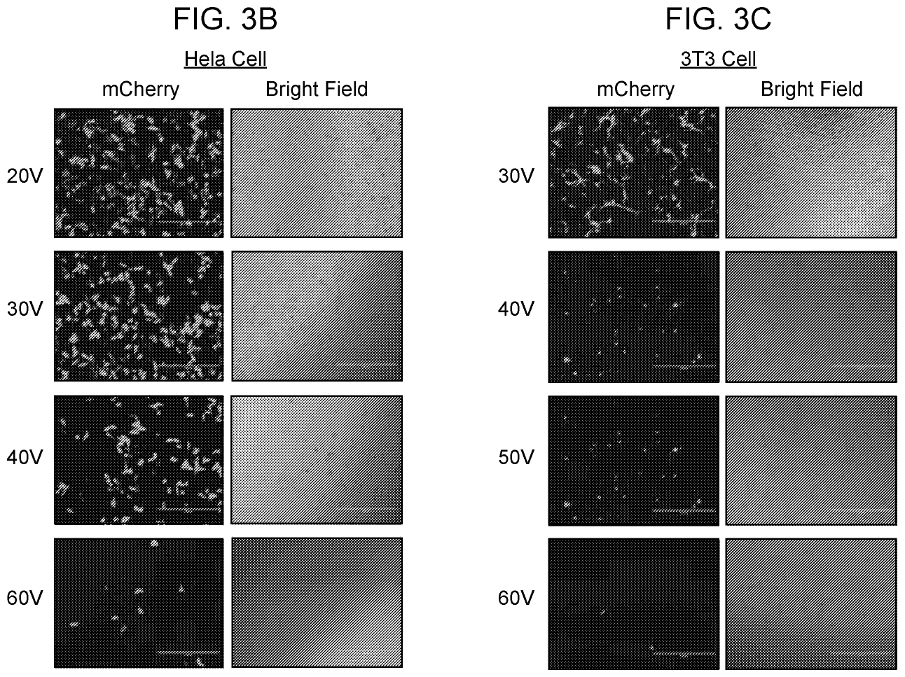

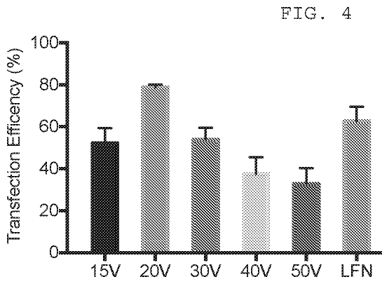

To test the delivery device for use in transfection, human embryonic kidney cells 293 (HEK293), HeLa cells, and NIH 3T3 fibroblast cells (3T3) were cultured overnight in the first reservoir that was bottom-sealed with a porous polycarbonate membrane. The cells were transfected with both mCherry encoded mRNA (FIGS. 2A-2C) and green fluorescent protein (GFP) encoded DNA plasmid (FIGS. 3A-3C). A range of applied voltages (e.g. from 15V to 80V) to create an electric field were tested. The voltage intensity ranging from 15V to 50V resulted in the highest transfection efficiencies. HEK293, HeLa, and 3T3 cells resulted in mRNA transfection efficiency of 85%, 95% and 75%, respectively. Since DNA plasmid transfection requires more cellular activities, DNA plasmid transfection efficiencies of the three types of cells were slightly lower than the mRNA efficiency: 65%, 90%, and 40%, respectively. The delivery device of the present disclosure was compared to LFN 2000-mediated delivery by analyzing the DNA plasmid transfection efficiencies with HeLa cells. The results from flow cytometry sorting (FACS) showed that the delivery device of the present disclosure resulted in at least a 20% higher yield than LFN system (FIG. 4).

### Highly Efficient Transfection of Nucleic Acids to Suspension Cells

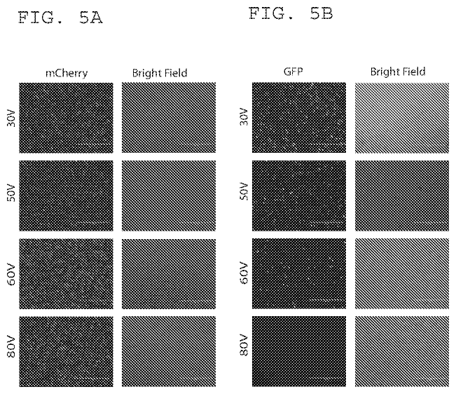

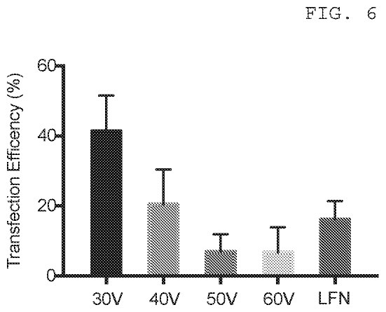

The delivery device of the present disclosure was also suitable for transfection of cells in suspension. Jurkat, a human T-cell lymphoma cell line, was used to test the transfection of nucleic acids into the cells due to the cell line's difficulty in being transfected. To deliver mRNA or a DNA plasmid into Jurkat cells using the delivery device of the present disclosure, Jurkat cells were centrifuged at 150 g for 3-5 minutes to allow the cells to come in physical contact with the surface of the porous membrane (FIGS. 1A-1C). A small amount (e.g. 1 μl to 5 μl) of mRNA or DNA plasmids were placed below the porous membrane in the second reservoir, and the biomolecules were dragged into the cells through the nanopores in the membrane by the applied electric forces generated by the electric field. Different voltage intensities were tested, ranging from 30V to 80V (FIGS. 5A-5B). Results showed that 30 V was sufficient for high efficiency delivery of mRNA or DNA plasmids into the cells. Cell image analysis showed that the transfection efficiency of mRNA and DNA plasmid resulted in 90% and 60% transfection efficiency, respectively. Furthermore, results from fluorescence-activated cell sorting (FACS) showed that the transfection efficiency from the delivery device of the present disclosure was 40% greater than LFN 2000-mediated transfection (FIG. 6).

### High Efficient Delivery of Proteins and Cas9-sgRNA Ribonucleoprotein Complexes (RNPs)

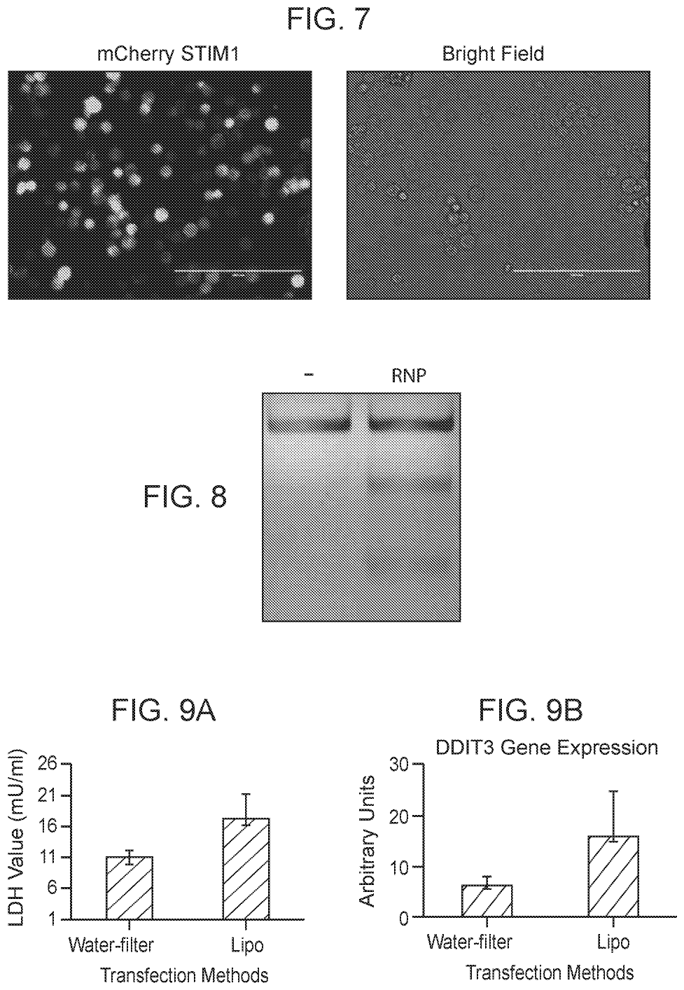

The efficiency of the delivery device in transporting biomolecules into the cells was tested by delivering mCherry-tagged protein STIM1 (98 kDa) or SpyCas9-sgRNA RNPs into cells for gene editing. The delivery process for both the protein and RNPs was exactly the same as for the delivery of nucleic acids. Results showed that the delivery efficiency of mCherry-tagged STIM1 protein into HEK293 cells was as high as 90% (FIG. 7). The delivery of SpyCas9-sgRNA RNP into HEK293 cells was also efficient as shown from a subsequent T7E1 assay. More than 50% of PPIB target DNAs were cut from the RNP (FIG. 8).

Water-filter Nanopore Delivery System Cause less Damage to the Delivered Cells

To determine if the transfection process using the delivery device of the present disclosure causes damage to the transfected cells, leakage of LDH in the culture media was measured and the expression profile of DNA damage inducible transcript 3 gene (DDIT3) was analyzed in transfected HeLa cells with qPCR. These assays demonstrated that transfection with the delivery device of the present disclosure was less toxic to the transfected cells than the LFN-mediated transfections (FIGS. 9A-9B).

While the present invention has been described with reference to the specific cases thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. A delivery device for delivering a biomolecule into a eukaryotic cell, the device comprising:
a first reservoir comprising a proximal end and a distal end;
a second reservoir comprising a proximal end and a distal end;
a porous membrane comprising at least one nanopore with a pore size ranging from about 50 nm to about 150 nm, wherein the at least one nanopore is fluidically connected to the first reservoir and the second reservoir; and
two or more electrodes configured to generate an electric field across the porous membrane.

2. The device of claim 1, wherein the at least one nanopore has a pore size of from 50 nm to about 100 nm.

3. The device of claim 2, wherein the at least one nanopore has a pore size of from 100 nm to about 150 nm.

4. The device of claim 1, wherein the porous membrane comprises a nanopore density ranging from 1×10⁸ nanopores per cm² to 5×10⁸ nanopores per cm².

5. The device of claim 1, wherein the porous membrane comprises a polymer material.

6. The device of claim 1, wherein the porous membrane comprises an elastomer, a thermoset, a thermoplastic, glass, quartz, or a silicon material.

7. The device of claim 5, wherein the material comprises polydimethylsiloxane (PDMS), polyimide, polyurethane, SU-8, polymethylmethacrylate (PMMA), polycarbonate (PC), polystyrene (PS), polyethylene terephthalate (PET), polyvinylchloride (PVC)), or polycaprolactone (PCL).

8. The device of claim 1, wherein the two or more electrodes comprise a first electrode and a second electrode.

9. The device of claim 8, wherein the first electrode is positioned at the distal end of the first reservoir and the second electrode is positioned at the distal end of the second reservoir.

10. The device of claim 1, wherein the device has an overall area of from about 0.01 cm² to about 15 cm².

11. The device of any one of claims 1-10, wherein the thickness of the porous membrane ranges from 10 μm to 100 μm.

12. The device of any one of claims 1-11, wherein the two or more electrodes are two or more platinum or titanium electrodes.

13. A method of delivering a biomolecule into a eukaryotic cell, the method comprising:
applying an electric field across the porous membrane of the delivery device of any one of claims 1-12,
wherein the biomolecule is present in a liquid medium in the second reservoir,
wherein the eukaryotic cell is present in a liquid medium in the first reservoir and is in physical contact with the porous membrane, and
wherein application of the electric field provides for delivery of the biomolecule into the eukaryotic cell.

14. The method of claim 13, further comprising centrifuging the eukaryotic cell present in the first reservoir of the delivery device before applying the electric field.

15. The method of claim 14, further comprises culturing the at least one eukaryotic cell at a proximal end of the first reservoir for a period of time to allow the at least one eukaryotic cell to contact the porous membrane.

16. The method of any one of claims 13-15, wherein the electric field comprises a voltage ranging from 15 volts to 80 volts.

17. The method of claim 16, wherein the electric field comprises a voltage ranging from 50 volts to 80 volts.

18. The method of any one of claims 13-17, wherein the biomolecule is selected from the group consisting of a DNA, an RNA, a polypeptide, ribonucleoprotein (RNP), and a deoxyribonucleoprotein (DNP), and combinations thereof.

19. The method of claim 18, wherein the RNA is a single-molecule CRISPR/Cas effector peptide guide RNA.

20. The method of claim 19, wherein the RNP comprises a CRISPR/Cas effector polypeptide and a guide RNA.

21. The method of any one of claims 13-20, wherein the first reservoir comprises a population of eukaryotic cells, and wherein the biomolecule is delivered into at least 50% of the population of eukaryotic cells.

22. The method of any one of claims 13-21, wherein at least 50% of the population of eukaryotic cells remains viable following application of the electric field.
